(19) United States
(12) Patent Application Publication
(10) Pub. No.: US 2005/0204433 A1
(43) Pub. Date: Sep. 15, 2005

(54) Method for recombinating plastid using procaryotic recombinase gene

(76) Inventors: Jang-Ryol Liu, Daejeon (KR); Won-Joong Jeong, Daejeon (KR); Sung-ran Min, Daejeon (KR); Seok-won Jbong, Daejeon (KR); Su-kyoung Han, Daejeon (KR)

Correspondence Address: STAAS & HALSEY LLP, SUITE 700, 1201 NEW YORK AVENUE, N.W., WASHINGTON, DC, 20005, US

(21) Appl. No.: 10/500,664

(22) PCT Filed: Dec. 31, 2002
(86) PCT No.: PCT/KR02/02506

§ 371 Date: May 20, 2005

(30) Foreign Application Priority Data
Jan. 3, 2002 (KR) 10-2002-0000218

(51) Int. Cl. A01H 1/00; C12N 15/82

(52) U.S. Cl. 800/294; 435/468

(57) ABSTRACT

The objective of this invention is to enhance the efficiency of plastid transformation using nuclear transformed plants in which the microbial recombinase A(recA) is to target to (or expressed in) the plastid. This invention will be better explained by the following detailed descriptions. A plant is transformed with a nuclear transformation vector containing the microbial recA gene added with a plastid targeting sequence. In this nuclear transformed plant, the frequency of plastid transformation is enhanced greater than two-folds due to increased homologous recombination between the plastid transformation vector carrying genes of interest (or target genes) and the plastid genome. In addition, because plastid transformation is accomplished through a gradual process, adventitious shoots selected after being subjected to plastid transformation should be cut into explants, and then shoots regenerated from the explants are to be reselected until all of the plastids in the shoots are uniformly transformed. However, when the nuclear transformed plant is used, the number of reselection is reduced to ½ to ⅓ due to increased homologous recombination.

### TECHNICAL FIELD

The present invention relates to a method for enhancing efficiency of plastid transformation by using microbial recombinase, and more particularly to a method comprising the steps of (a) transforming a nucleus of a plant with a recombinant expression vector containing a microbial (a prokaryote) recombinase gene and a targeting sequence of a plastid; (b) selecting a plant transformant expressing recombinase in the plastid at high level and (c) retransforming the plant transformant with a plastid transformation vector containing a nucleotide sequence of a target gene and a selective marker gene, respectively.

### BACKGROUND ART

Plastids are classified according to their functional roles such as chloroplasts which are involved in photosynthesis, amyloplasts which store starch, leukoplasts which do not contain pigments, and chromoplasts which give colors to flowers and fruits. In general, a plant cell can contain as many as 200 plastids and each plastid has about 100 genomes which leads to a total of 10,000 ˜ 50,000 copies of genes per each plant cell. However, a single nucleus of a plant normally contains 1 ˜ 2 genomes on average.

Therefore, a target protein may be expressed more effectively if an exogenous gene is introduced by a plastid transformation, by approximately 10,000 folds, when compared theoretically with a case of a simple nuclear transformation.

Recently, in line with the above theoretical concept, there was disclosed a method for rendering new traits on plants, where an exogenous gene was inserted into a plant plastid genome by plastid transformation (Svab, et al., 1990; Staub, et al., 2000). The method largely consists of two major steps: (a) transforming a plastid; and (b) selecting an appropriate plant transformant.

Specifically, the plastid transformation can be accomplished by homologous recombination, where typical nucleotide sequences of a plastid, exploited as a border for homologous recombination, are ligated to an exogenous gene and then introduced by means of particle bombardment.

Then, after the plastid transformation, plant cells are allowed to re-differentiate after 2 ˜ 7 rounds of screenings attempted for the purpose of acquiring homoplasmy of all the plastids in a cell. In the absence of the above screening process, the plastid in a cell will be transformed only in part and thus the plant will gradually lose transformed plastids as development proceeds.

Most researches on plastid transformation have been attempted by using tobacco plants with a few other successes reported in Arabidopsis, potato, tomato and the like. However, plants other than tobacco plants are known very ineffective in terms of plastid transformation. The inefficiency in the plastid transformation appears largely due to the relatively low frequency of plastid transformation, a relatively long period of time to be homoplasmy and a complex work required for screening plant transformants. In case of tobacco plants, however, the elucidation of its characteristics was made possible after enormous and extensive studies thus enabling relatively high efficiency of transformation rate.

One way to overcome the above-mentioned difficulties may be to enhance a frequency of homologous recombination in a plastid.

Recombinase has been known to be involved in homologous recombination. Further, there have been reported a 10-fold increase in the frequency of homologous recombination in a nucleus of a cell when Escherichia coli recombinase was expressed in nuclei of microorganisms, higher plant cells of tobacco or animal cells (Stohl and Seifert, 2001; Bakhlanova, et al., 2001; Reiss, et al., 1996; 1997; Shcherbakova, et al., 2000; Vispe, et al., 1998).

Therefore, it is in high demand to develop a new method for enhancing the efficiency of plastid transformation and reducing the time required for screening the homoplasmic plastid transformants.

Accordingly, the inventors of the present invention have attempted to solve the aforementioned problems of the conventional techniques. As a result, they discovered a novel method for plastid transformation with high frequency of transformation as well as homologous recombination, wherein the steps of which comprise: utilization of a plant for the transfer of recombinase introduced into a nucleus in a plastid; construction of a vector for plastid transformation which contains a nucleotide sequence of a target gene and a selective marker gene; transformation of a plastid; and selection of appropriate transformants according to the level of expression of the marker gene(s) in the plastid.

### BRIEF DESCRIPTION OF DRAWINGS

The above and other objects, features and other advantages of the present invention will be more clearly understood from the following detailed description taken in conjunction with the accompanying drawings, in which;

FIG. 1 depicts a process for constructing a vector for nuclear transformation of a plant of the present invention;

FIG. 2 depicts experimental result of northern blot of the nuclear transformant of a plant;

FIG. 3 depicts a process for constructing a vector for plastid transformation of the present invention;

FIG. 4 depicts efficiencies of the transformation of the present invention.

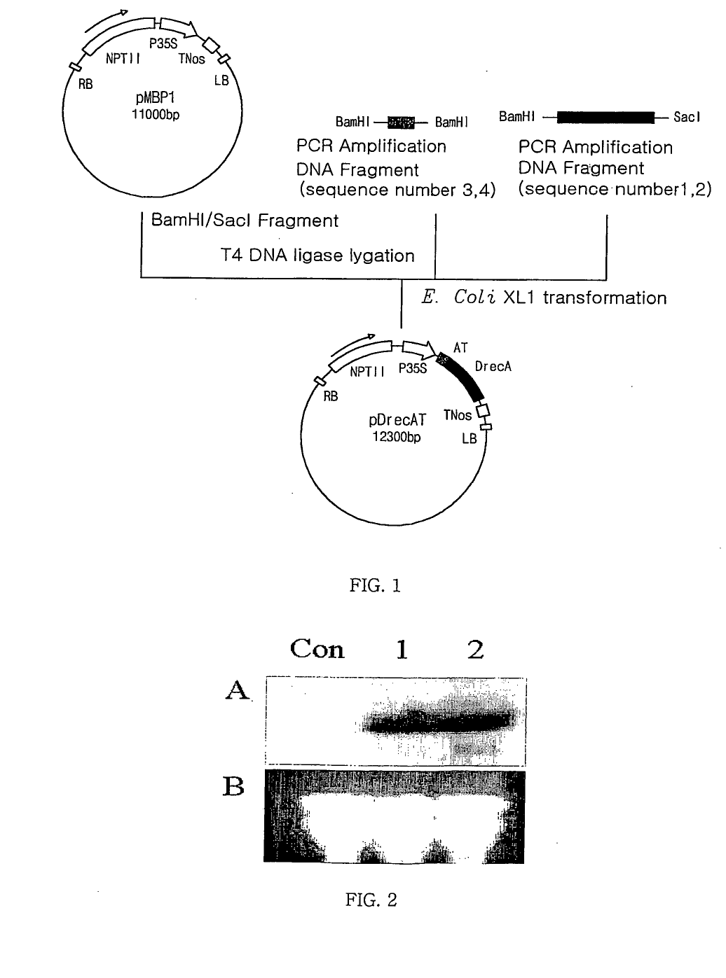

### DETAILED DESCRIPTION OF THE INVENTION

The present invention relates to a method for plant transformation which can accomplish homologous recombination with high efficiency by a simple manipulation and transform a plastid effectively by exploiting a plant with recombinase in a plastid.

In order to attain the above-mentioned object, the present invention provides a method for plastid transformation comprising the following steps of:
(a) constructing a recombinase expression vector for nuclear transformation of a plant which contains a nucleotide sequence of a recombinase gene active in a plastid and a targeting sequence for a plastid;
(b) preparing a primary plant transformant, wherein a nuclear transformed plant is prepared by using the recombinase expression vector;
(c) constructing a plastid transformation vector of a plant plastid which contains at least one nucleotide sequence of a target gene and a selective marker gene, respectively, which can be expressed in the plastid; and
(d) preparing a secondary plant transformant from the primary plant transformant obtained in (b) by using the plastid transformation vector.

Besides, in order to attain the above-mentioned object, the present invention provides a method for enhancing the efficiency of plastid transformation, which uses a plant transformant already transformed by a recombinase gene active in a plastid by using a similar method.

Specifically, the present invention provides a method for transforming a plastid which comprises the following steps of:
(a) constructing a plastid vector for transformation of a plant plastid which contains at least one nucleotide sequence of a target gene and a selective marker gene, respectively, which can be expressed in the plastid; and
(b) preparing a secondary plant transformant from said plant transformed by a recombinase gene active in a plastid by using the plastid transformation vector.

Namely, the present invention provides a method for enhancing efficiency of plastid transformation, which uses a plant transformant (the primary plant transformant) wherein recombinase derived from a prokaryote can be transferred to a plastid and be active in the plastid to be transformed by a plastid transformation vector containing both an exogenous gene and a selective marker gene.

Any recombinase active in a plastid of a higher plant can be used in the present invention. Specifically, the recombinase can be selected from the group consisting of Deinococcus radiodurans recA, E. coli recA, and the like.

Any targeting sequence which can transfer recombinase to a plastid can be used in the present invention as a targeting sequence. Specifically, the targeting sequence can be selected form the group consisting of Rubisco small subunit, AGPase, chlorophyll AB binding(Cab) protein and the like.

Any exogenous gene can be inserted into the plastid transformation vector of the present invention, regardless of its kind, as long as an exogenous trait to be introduced into a plant cell can be expressed by the gene. Specifically, genes such as BT toxin (Bt) gene, herbicide (bar, glyphosate) resistant gene, somatotropin and the like can be used alone or in combination depending on circumstances.

Any selective marker gene can be inserted into the plastid transformation vector of the present invention, if it has particular physicochemical characteristics sufficient to distinguish a secondary plant transformant from a plant without secondary transformation. Specifically, the selective marker gene can be selected from the group consisting of (1) genes for 16S subunit of ribosome resistant to spectinomycin or streptomycin; (2) genes for proteins resistant to antibiotics such as spectinomycin, streptomycin, kanamycin and the like; (3) genes for enzymes such as cytosine deaminase, betaine aldehyde dehydrogenase (BADH) and the like; and/or (4) genes for green fluorescence protein (GFP) and they can be used alone or in combination thereof. In particular, it is preferred that GFP gene be used with other selective marker genes to afford physical identification of secondary plant transformants. It is more preferred that other selective marker genes and GFP gene be connected in an operon so that only plant transformants with transformed plastid can grow on a selective medium while during which homologous recombination is visually distinguished. Further, it is noteworthy that a plant containing GFP in a plastid emits green fluorescence when exposed to a long-wave UV light.

### EXAMPLES

This invention is further illustrated by the following examples. However, these examples should not be construed as limiting the scope of this invention in any manner.

In the following Examples, spectinomycin resistance gene and GFP gene were used together as selective marker genes, but it is also possible that other selective marker gene is used alone or in combination with GFP gene. Moreover, the subject plant for transformation in the present invention is not limited to tobacco plants but it can be extended to other plants. Besides, altbeit a plant transformant (the primary plant transformant) containing microbial recombinase in a plastid is prepared directly in the present invention, any plant transformants already constructed for other purposes may be also used.

However, it will be appreciated that those skilled in the art, on consideration of this disclosure, may make modifications and improvements within the spirit and scope of the present invention.

The transformation vectors of the present invention have not been deposited since they can be manufactured easily by those skilled in the art.

### EXAMPLE 1

### Construction of a Nuclear Transformation Vector Containing Recombinase Gene

In order to prepare a plant transformant with active recombinase in a plastid, a nuclear transformation vector for plants which contain microbial recombinase gene and can be transferred to a plastid was constructed.

A targeting sequence of Arabidopsis putative recA and a recombinase gene from Deinococcus radiodurans recA were cloned respectively, ligated together, and then inserted to a BamHI/SacI restriction site located between 35S promoter and nos terminater. As a result, the nuclear transformation vector pDrecAAT for plants was obtained.

More specifically, DNA was isolated from a strain of Deinococcus radiodurans (Accession No: ATCC 13939) and then a DNA fragment of recA gene of 1.1 kb in size was cloned by PCR, which was performed 30 cycles by using the above DNA as a template and adding two primers of SEQ ID NOs 1 and 2 in the presence of PWO polymerase (BM Co.), wherein each cycle was proceeded under the condition of denaturing at 94° C., 1 minute, annealing at 55° C., 1 minute, and polymerization at 72° C., 60 seconds. Thus cloned gene was then ligated into a BamHI/SacI restriction site located between 35S promoter and nos terminater.

Meanwhile, a DNA fragment of a targeting sequence of a plastid of 0.2 kb in size was also cloned from Arabidopsis genomic DNA by PCR, which was performed 30 cycles by using the above DNA as a template and adding two primers of SEQ ID NOs 3 and 4 in the presence of PWO polymerase (BM Co.), wherein each cycle was proceeded under the condition of denaturing at 94° C., 1 minute, annealing at 55° C., 1 minute, and polymerization at 72° C., 60 seconds. Thus cloned gene was then ligated into a BamHI restriction site located between 35S promoter and recA. As a result, the plant nuclear transformation vector which is designed to transfer recombinase protein of Deinococcus radiodurans toward a plastid was obtained.

### EXAMPLE 2

### Preparation of Plant Transformant Containing Microbial Recombinase in Plastid

The nuclear transformation vector constructed in Example 1 was introduced to transform a plant primarily, which can be performed by well-known conventional methods or other advanced methods for transforming plants. Specifically, the Agrobacterium co-culture method was used for the experiment of the present invention.

The nuclear transformation vector prepared in Example 1, was introduced to Agrobacterium (GV3101 strain) by using the freeze thaw method, cultured in YEP medium containing 50 mg/L kanamycin and 50 mg/L rifampicin for 2 days, and then utilized to transform tobacco. Leaf explants of Nocotiana tabacum cv. Samsun cultured in sterile condition were floated on 10 mL of MS liquid medium (Murashige and skoog, 1962) were added with 200 μL of Agrobacterium suspension cells incubated for 2 days, and then they were co-cultured for 2 days. Further, Agrobacterium was washed with sterile distilled water and cultured on MS solid medium containing 100 mg/L kanamycin, 300 mg/L craforan, 2 mg/L BAP, 0.1 mg/L NAA at 25° C., at 2,000 lux so as to produce redifferentiated shoots.

After culturing for 3 ˜ 4 weeks, shoots generated on a selective medium were transferred to MS solid medium containing 300 mg/L craforan, 100 mg/L kanamycin to induce growth of roots, transferred again to soil and then cultured in a green house for next generations.

In order to identify the presence of the insertion and the expression of recombinase derived from Deinococcus radiodurans in primary plant transformant of the present invention, total RNAs were isolated from leaves of the plant transformant and then examined by northern blot analysis (See FIG. 2). In FIG. 2, lane Con denotes a plant without transformation; lane 1 and 2, plant transformants expressing recombinase; lane A, recombinase RNAs in northern blot; and lane B, loaded total RNAs.

Here, the primary plant transformant can be used in its transformed state or its progeny can be also used as an alternative.

### EXAMPLE 3

### Construction of Plastid Transformation Vector Containing GFP Gene

A plastid transformation vector which can easily identify a given plant whether it is transformed or not by visual inspection under UV exposure was prepared. More specifically, plastid transformation vector was constructed by referring to the pSBL-ctV2 for dicistronic expression of the aadA and gfp genes under the control of the plastid rrn promoter and was named pTIG.

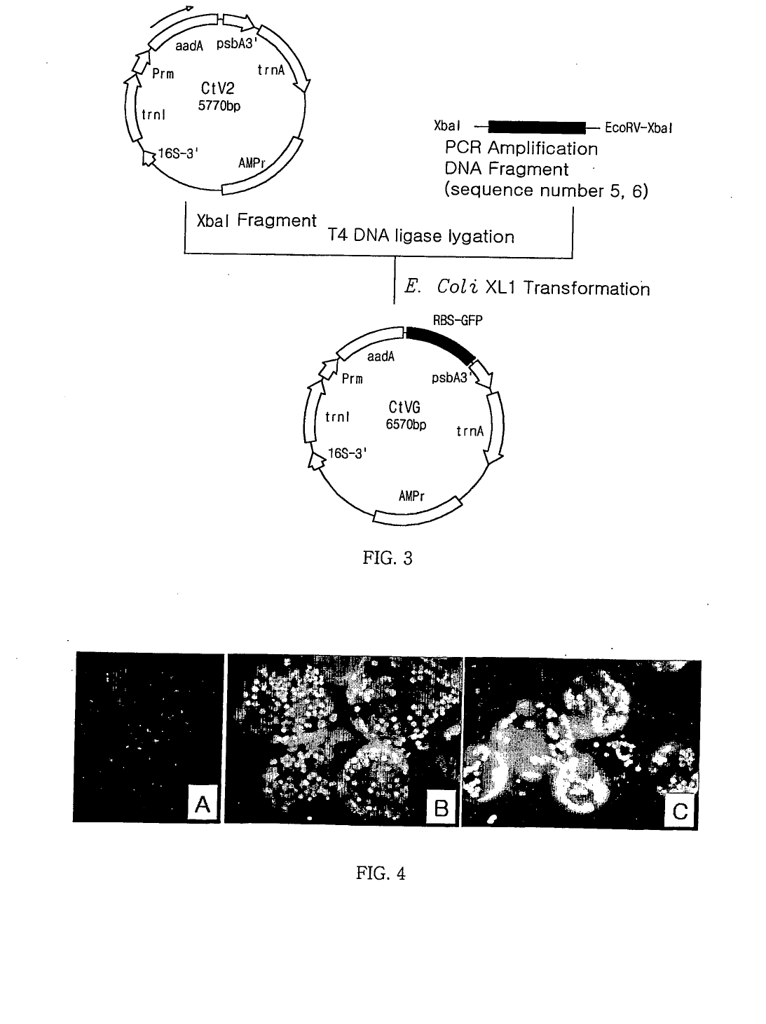

More specifically, in order to express mGFP4 gene, a GFP gene variant, primers were designed to include a ribosome binding site of SEQ ID NO 5 (AGGAGGTATAACA) at an upstream region of start codon and a DNA fragment of GFP gene was cloned by PCR, which was performed 30 cycles by using the above DNA as a template and adding two primers of SEQ ID NOs 5 and 6 in the presence of PWO polymerase (BM Co.), wherein each cycle was proceeded under the condition of denaturing at 94° C., 1 minute, annealing at 55° C., 1 minute, and polymerization at 72° C., 40 seconds. Thus cloned gene was then ligated into a downstream region of aadA gene, a spectinomycin resistant gene, and as a result, the plastid transformation vector pTIG in which GFP gene can be expressed from operon was constructed (See FIG. 3).

### EXAMPLE 4

### Plastid Transformation By Particle Bombardment

Progeny of plant transformants prepared in Example 2 and a control group without a nuclear transformation were attempted for experimental transformations according to the present invention by using the vector constructed in Example 3.

The nuclear transformation plant and the control group plant were germinated in a sterile condition for 8 weeks, respectively. Leaves of young plants were detached and placed on MS medium containing 1 mg/L BAP, 0.1 mg/L NAA and then exploited to the plastid transformation.

The plastid transformation vector pTIG was coated with gold particles having a radius of 0.6 μm in size and then used to transform a plastid by using PDS-1000/He gene delivery system purchased from BioRad Co. Ltd. under a condition of 1,100 psi acceleration power, 9 cm target distance and 28 in/Hg of vacuum. Then, the resultants were cultured in a dark room at 25° C. with 2,000 lux for 2 days. Explants of tobacco leaves cut into sections approximately 2-5 mm square, incubated in MS medium containing 1 mg/L BAP, 0.1 mg/L NAA, 500 mg/L spectinomycin and the plastid transformants were selected.

### EXAMPLE 5

### Examination of Efficiency for Transforming a Plastid in Tobacco

In the secondary plant transformant prepared above, in which the plastid transformation vector pTIG is inserted into a plant expressing recombinase from a plastid, the efficiency for transforming a plastid was investigated.

The plant cell with untransformed plastids appeared red, autofluorescence of chlorophyll under UV. On the other hand, in the plant where the plastid was transformed, the plant cell with transformed plastids showed varying fluorescences from reddish-yellow to green fluorescence under UV, depending upon the level of GFP expression. The control group (which was not transformed by microbial recombinase) was compared to estimate whether a plastid be transformed or not and to measure the transformation efficiency. As a result, it was confirmed that the efficiency for transforming a plastid becomes higher when the microbial recombinase is exploited.

More specifically, total petridishes, which were selected by the primary screening after transformation (namely, cultured for 4 weeks) in Example 4, were examined by collecting petridishes having redifferentiated shoots with green fluorescence with respect to the transformation efficiency. As a result, it was confirmed that the plant transformants with microbial recombinase in their plastids have greater efficiency of transformation than the control group by more than two-folds (See Table 1).

Table 1

Efficiency of plastid transformation

| | | Plant transformant of |
|---|---|---|
| | Control group | the present invention |
| Transformation rate | 5/11 (45.4%) | 8/9 (88.9%) |
| (efficiency %) | | |

Transformation Rate =Number of Petri dishes showing Green Fluorescence/Number of Bombardment

Furthermore, protoplasts were isolated from transformed shoots screened for 4 weeks and plastids expressing GFP in cells were observed under a fluorescence microscope so as to calculate the efficiency of homologous recombination. Also, the plant transformant of the present invention (the plant transformant containing microbial recombinase which has undergone secondary transformation) was verified to produce still greater amount of GFP, compared with the control group (the plant transformant not containing microbial recombinase which has undergone secondary transformation) (See FIG. 4). In FIG. 4, lane A denotes cells of tobacco explant which was untransformed; lane B, the control group; and lane C, cells of tobacco explant obtained by the process of the present invention.

The level of GFP expression in the plant transformant of the present invention selected after the primary screening was similar to that of the control group, which were selected after 2 ˜ 3 rounds of selection procedure. Consequently, it was confirmed that the method of plastid transformation of the present invention, wherein the plant transformant containing microbial recombinase was re-transformed, remarkably enhance the rate of homologous recombination, as compared with the conventional method as depicted in the control group.

### INDUSTRIAL APPLICABILITY

As demonstrated and confirmed above, the present invention relates to a method for enhancing the efficiency of plastid transformation by using a nuclear transformed plant containing microbial recombinase in a plastid and a method for enhancing the efficiency of homologous recombination, which can reduce a period of time required to prepare homoplasmy and extend its applications to other plants which have been suffering from low efficiency or unfeasibility of plastid transformation in addition to tobacco. The methods of the present invention can be useful to express and collect industrial exogenous proteins from various plants.

Furthermore, the methods of the present invention can increase the efficiency of homologous recombination still more remarkably than conventional methods and reduce the number of reselection steps down to the level of ½˜ ⅓ of the original. Therefore, the plastid transformed plant can be prepared successfully with more than 2-fold increase.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for transforming a plant plastid which the following steps of:
A. constructing a recombinase expression vector for nuclear transformation of a plant which contains a targeting sequence for a plastid and a nucleotide sequence of a recombinase protein active in a plastid;
B. preparing a primary plant transformant, wherein a nuclear transformed plant is prepared by using said recombinase expression vector;
C. constructing a plastid transformation vector which contains at least one nucleotide sequence of a target gene and a selective marker gene, respectively, which can be expressed in said plastid; and
D. preparing a secondary plant transformant from said primary plant transformant obtained in (b) by using said plastid transformation vector.

2. The method for transforming a plant plastid according to claim 1, wherein said recombinase gene is derived from a prokaryote.

3. The method for transforming a plant plastid according to claim 1, wherein said selective marker is selected from the group consisting of 16S subunit of a ribosome resistant to spectinomycin or streptomycin; a protein resistant to antibiotics such as spectinomycin, streptomycin, kanamycin and the like; an enzyme such as cytosine deaminase, betaine aldehyde dehydrogenase (BADH) and the like; and/or green fluorescence protein (GFP).

4. A method for transforming a plant plastid which comprises the following steps of:
(a) constructing a plastid transformation vector which contains at least one nucleotide sequence of a target gene and a selective marker gene, respectively, which can be expressed in said plastid; and preparing a secondary plant transformant from said plant transformed by a recombinase gene active in a plastid by using said plastid transformation vector.

5. The method for transforming a plant plastid according to claim 4, wherein said selective marker is selected from the group consisting of 16S subunit of a ribosome resistant to spectinomycin or streptomycin; a protein resistant to antibiotics such as spectinomycin, streptomycin, kanamycin and the like; an enzyme such as cytosine deaminase, betaine aldehyde dehydrogenase (BADH) and the like; and/or green fluorescence protein (GFP).

6. The method for transforming a plant plastid according to claim 5, wherein said recombinase gene is derived from a prokaryote.
